(19) United States
(12) Patent Application Publication
(10) Pub. No.: US 2012/0113411 A1
(43) Pub. Date: May 10, 2012

(54) OPTICAL FIBER SENSORS

(75) Inventors: Youzhi Li, Longmont, CO (US); Edward M. McKenna, Boulder, CO (US); Andy S. Lin, Boulder, CO (US)

(73) Assignee: Nellcor Puritan Bennett LLC, Boulder, CO (US)

(21) Appl. No.: 12/942,135

(22) Filed: Nov. 9, 2010

(51) Int. Cl. G01N 33/48; G01N 21/00; B23P 11/00; G01J 3/42

(52) U.S. Cl. 356/41; 356/319; 356/432; 29/428

(57) ABSTRACT

The present disclosure describes the use and the manufacture of a fiber optic sensor having an angled terminal portion of a fiber optic element. In one embodiment, an optical fiber is cut at an angle and a portion of the fiber's jacket is removed near the angle so that the cladding is exposed. Light may then travel through the fiber, reflect off the angled portion, and emit through the exposed cladding into a patient. Light may also be collected from the patient using the same or a different fiber optic element having an angled terminal portion. In one embodiment, the emitted light can then be collected and analyzed to derive various physiological parameters. In certain embodiments, the fiber optic sensor may be used in environments where metallic and/or electronic sensors are not suitable.

### BACKGROUND

The present disclosure relates generally to medical diagnostic sensors and, more particularly, to energy efficient spectrophotometric sensors.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor and sense certain physiological characteristics of their patients. Accordingly, a wide variety of devices have been developed for monitoring and sensing many such physiological characteristics. One category of monitoring and sensing devices includes spectrophotometric monitors and sensors. This category of device typically measures the absorption and/or reflection of energy at particular, discrete wavelengths within the electromagnetic spectrum and may allow one or more physiological parameters to be determined based upon these measurements. Such devices provide doctors and other healthcare personnel with the information they need to provide the best possible healthcare for their patients. As a result, such monitoring and sensing devices have become an indispensable part of modern medicine.

One technique for monitoring certain physiological characteristics of a patient using spectrophotometric devices is commonly referred to as pulse oximetry, and the devices built based upon pulse oximetry techniques are commonly referred to as pulse oximeters. Pulse oximetry may be used to measure various blood flow characteristics, such as the blood-oxygen saturation of hemoglobin (SpO₂) in arterial blood, the volume of individual blood pulsations supplying the tissue, and/or the rate of blood pulsations corresponding to each heartbeat of a patient. In fact, the “pulse” in pulse oximetty refers to the time varying amount of arterial blood in the tissue during each cardiac cycle.

Pulse oximeters typically utilize a non-invasive sensor that transmits light through a patient's tissue and that photoelectrically detects the absorption and/or scattering of the transmitted light in such tissue. One or more of the above physiological characteristics may then be calculated based upon the amount of light absorbed and/or scattered. More specifically, the light passed through the tissue is typically selected to be of one or more wavelengths that may be absorbed and/or scattered by the blood in an amount correlative to the amount of the blood constituent present in the blood. The amount of light absorbed and/or scattered may then be used to estimate the amount of blood constituent in the tissue using various algorithms. This determination may be performed in a monitor coupled to the sensor that receives the necessary data for the blood constituent calculation.

Conventional pulse oximeter sensors have a multitude of electronic components that are part of the sensor, for example light emitting diodes (LEDs), resistors, capacitors, inductors, and so forth. Metals are also typically contained in the sensor as part of the wiring, the electronic components, the sensor body, and in other sensor components. However, electronic devices, as well as devices containing metallic components, may be unsuitable for use in certain medical environments. For example, electronic devices, such as pulse oximetry sensors, may be unsuitable for use inside magnetic resonance imaging (MRI) scanners where the presence of electromagnetic fields and/or magnetic materials may result in distortion or degradation of the image being created.

### BRIEF DESCRIPTION OF THE DRAWINGS

Advantages of the disclosed techniques may become apparent upon reading the following detailed description and upon reference to the drawings in which:

FIG. 1 illustrates components of a monitoring system, in accordance with one embodiment of the present disclosure;

FIG. 2 depicts a terminal portion of a light fiber used to transmit light, in accordance with one embodiment of the present disclosure;

FIG. 3 depicts a terminal portion of a light fiber used to acquire light, in accordance with one embodiment of the present disclosure;

FIG. 4 illustrates a sensor placement for a transmittance sensor, in accordance with one embodiment of the present disclosure;

FIG. 5 illustrates a sensor placement for a reflectance sensor, in accordance with one embodiment of the present disclosure, and;

FIG. 6 illustrates a block diagram of a spectrophotometric system, in accordance with one embodiment of the present disclosure.

### DETAILED DESCRIPTION OF SPECIFIC EMBODIMENTS

One or more specific embodiments of the present invention will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The present disclosure describes a fiber optic sensor that may be used inside environments that are unsuitable for devices containing metal or electronic circuitry. In particular, embodiments of the present disclosure are typically free of electronics or metals as part of the sensor body. Instead, as discussed herein, such sensors may utilize fiber optic waveguides to transmit light into and out of such sensitive environments, such as the imaging bore of a magnetic resonance imaging (MRI) system.

Present embodiments may apply to a variety of spectrophotometric sensors, for example, pulse oximetry sensors. Moreover, as disclosed herein, the data of interest that may be observed using a spectrophotometric sensor may similarly vary depending on the capabilities of each device. For example, a pulse oximetry sensor may transmit absorption data that may be used to derive measurements of pulse rate, blood oxygen saturation, and/or total hemoglobin, and so forth. Because the embodiments presently disclosed may eliminate the need for the electronics and the metals found in conventional sensors, fiber optic sensors may be used in a wide variety of medical environments, including MRI scanners.

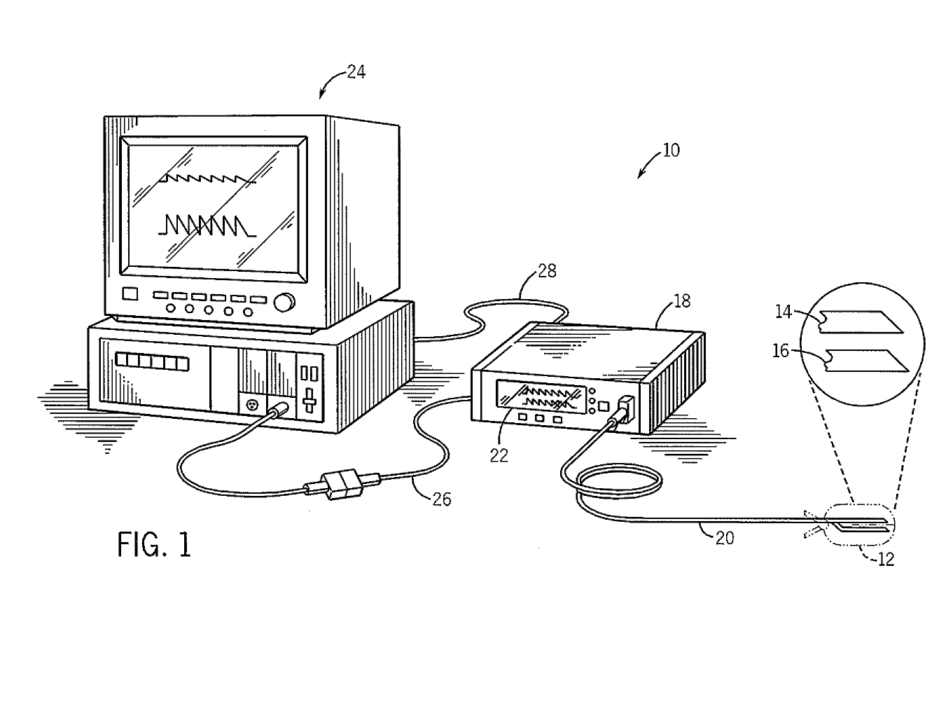

With the foregoing in mind, FIG. 1 depicts an embodiment of a spectrophotometric medical diagnostic sensor system 10 that may emit and collect light through a sensor 12 (depicted in outline form) having a body housing fiber optic elements, such as terminal portions 14 and 16 of respective fiber optic elements. The sensor 12 may be formed so as to position the terminal portions 14 and 16 of the fiber optic elements proximate to or adjacent to the patient tissue when the sensor 12 is applied. For example, in one embodiment, the sensor 12 may include a clip-style sensor body. In other embodiments, the sensor 12 may include a bandage-style sensor body. In an embodiment in which the sensor 12 is a bandage-style sensor, the sensor may include stiffening or reinforcing elements that provide a suitable degree of rigidity such that the fiber optic components remain in a suitable orientation or configuration with respect to a patient's tissue when the sensor 12 is applied.

In one embodiment, the sensor 12 does not include metallic, magnetic, and/or electronic components. For example, the sensor 12 may be formed from a polymeric composition (e.g., plastics, thermoplastics, or other polymeric materials, such as polyurethane, polypropylene, nylon, and so forth), though other suitable non-metallic and/or non-magnetic materials may also be used. The sensor 12 may also include or be formed using a thermoplastic elastomer or other conformable material. In such embodiments, the thermoplastic elastomer may include compositions such as thermoplastic polyolefins, thermoplastic vulcanizate alloys, silicone, thermoplastic polyurethane, as well as other suitable non-metallic and/or non-magnetic compositions.

The system 10 may include a patient monitor 18 that is connected to the sensor 12 via a fiber optic connection 20. The patient monitor 18 may include a display 22, a memory, a processor, and various monitoring and control features for processing data acquired via the sensor 12. Based on light received from the sensor 12, the patient monitor 18 may display patient diagnostic measurements and perform various additional algorithms. For example, when the system 10 is configured for pulse oximetry, the patient monitor may perform blood oxygen saturation calculations, pulse measurements, and other measurements based on the received light. Furthermore, to upgrade conventional operation provided by the monitor 18 to provide additional functions, monitor 18 may be coupled to a multi-parameter patient monitor 24 via a cable 26 connected to a sensor input port or via a cable 28 connected to a digital communication port, for example.

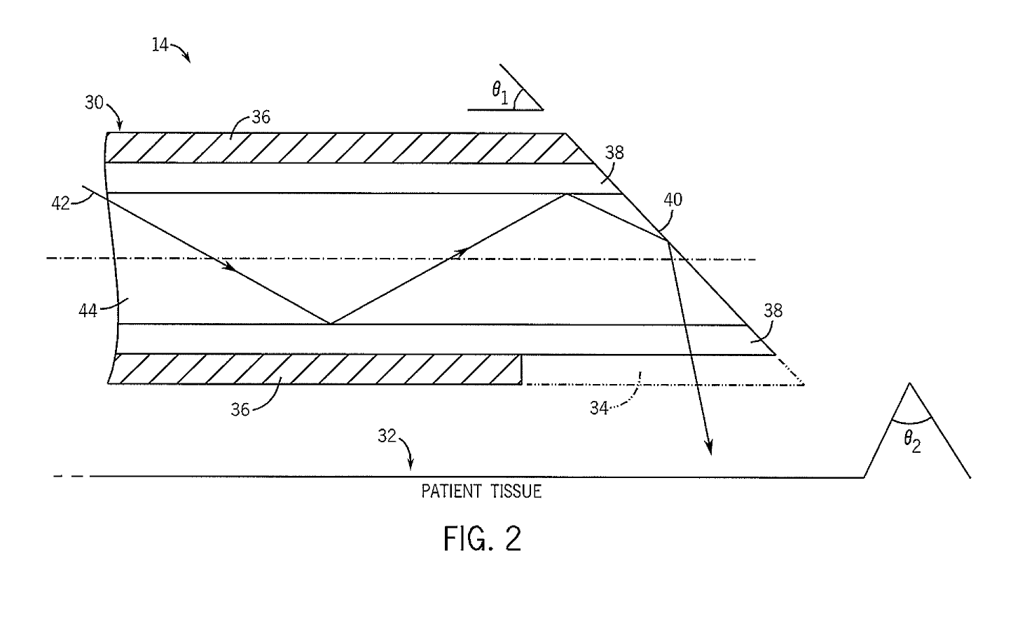

Turning to FIG. 2, the figure depicts a terminal portion 14 of a fiber optic element 30 of the sensor 12. In the depicted embodiment, the terminal portion 14 is used for light emission into a patient tissue 32. In this embodiment, the fiber optic element 30 may be a multi-mode plastic optical fiber in which the terminal portion 14 terminates at an angle, such as due to an angled cut. In another embodiment, the fiber optic element 30 may include multi-mode optical fiber bundles, each optical fiber in the bundle having an angled terminal portion 14. In other embodiments, any class of optical fiber that is capable of transmitting spectrophotometric light wavelengths may be used as the fiber optic element 30, for example, single-mode fiber. The angled terminal portion 14 may be used to reflect light traveling through the fiber outward towards the patient tissue 32.

The terminal portion 14 of the fiber optic element 30 may be manufactured by cutting the end of a fiber optic fiber at an angle of θ₁ and by removing a section 34 of the fiber's jacket 36 so as to expose the cladding 38. In one embodiment, the terminal portion 14 of the fiber optic element 30 may have an angle θ₁ of approximately 45°. However, in other embodiments, the angle θ₁ may be between 30° and 60°. Any number of methods may be used for cutting the fiber at an angle of θ₁. For example, ceramic scissors, an angled cutting jig, a laser cutter, a robotic fiber cutter, as well as other suitable fiber cutting techniques may be used to cut the terminal portion 14 at the angle θ₁.

In one embodiment, the end of the cut fiber may be polished to enhance the reflectivity of the boundary surface 40. In another embodiment, it may be coated or capped with a reflective layer so that the reflectivity of the boundary surface 40 is enhanced. Enhancing the reflectivity of the boundary surface 40 may allow for more emitted rays of light to reflect into the patient tissue 32. In yet another embodiment, the cut fiber may not need to be further polished or cut, based on the reflectivity of the boundary surface 40. That is, the cut may result in a boundary surface 40 having sufficient reflectivity so as to suitably reflect the rays of light into the patient tissue 32. Indeed, the polishing, coating, and/or capping of the boundary surface 40 may be based on the reflectivity already present in the boundary surface 40. That is, the boundary surface 40 may already have a certain amount of reflectivity, and so only minimal (or no) polishing, coating, and/or capping may be needed.

As discussed above, in one embodiment, a tissue facing portion of the fiber optic element 30 has a section 34 where the fiber's jacket 36 that has been removed, exposing the cladding 38. Any number of methods may also be used to remove the section 34 of the fiber's jacket 36. For example, hand-held cable strippers, hand-held cable slitters, a robotic stripper, a robotic slitter, as well as other jacket removal techniques may be used to remove the section 34 of the jacket 36 and to expose the cladding 38. In one embodiment, the exposed cladding 38 is placed up against the patient tissue 32 at section 34, physically contacting the patient tissue 32. In another embodiment, the exposed cladding 38 does not physically contact the patient tissue 32 and there is an air gap between the exposed cladding 38 and the patient tissue 32 at section 34. Additionally, a light coupling medium, such as a gel, may be used to enhance the coupling efficiency of the cut fiber with the patient tissue. Accordingly, the gel coating may be disposed on the ends of the cut fiber, approximately on the terminal portion 14 and/or terminal portion 16.

FIG. 2 also illustrates an example path that an emitted ray of light 42 may follow while traveling from the monitor 18 (shown in FIG. 1) through the fiber's core 44 into the patient tissue 32. The emitted ray of light 42 may reflect off the cladding 38 a multitude of times while traveling through the core 44 until it reaches the boundary surface 40. The boundary surface 40 is angled at an angle of θ₁ such that the ray of light 42 is reflected toward the patient tissue 32. The ray of light 42 may slightly refract as it passes through the cladding 38 before entering the patient tissue 32.

Emitted rays of light may enter the patient tissue 32 at an angle θ₂. The angle θ₂ may vary in embodiments having the boundary surface 40 coated or capped with a reflective layer and in embodiments having the boundary surface 40 left uncoated and uncapped. In certain embodiments, angle θ₂ may be derived based on the law of reflection of light and Snell's Law. Snell's law of refraction may take into account the refractive index of the core 44, the refractive index of the cladding 38, the refractive index of the environment (e.g., air) immediately adjacent to the terminal portion 14, and the refractive index of the patient tissue 32 (in embodiments where the exposed cladding 38 is physically contacting the patient) in order to derive angle θ₂. The angle θ₁ and/or the coating applied to the terminal portion 14 fiber optic element 30 may be selected taking into account the law of reflection and/or Snell's law to provide a desired angle θ₂ for entry of light into the patient tissue 32.

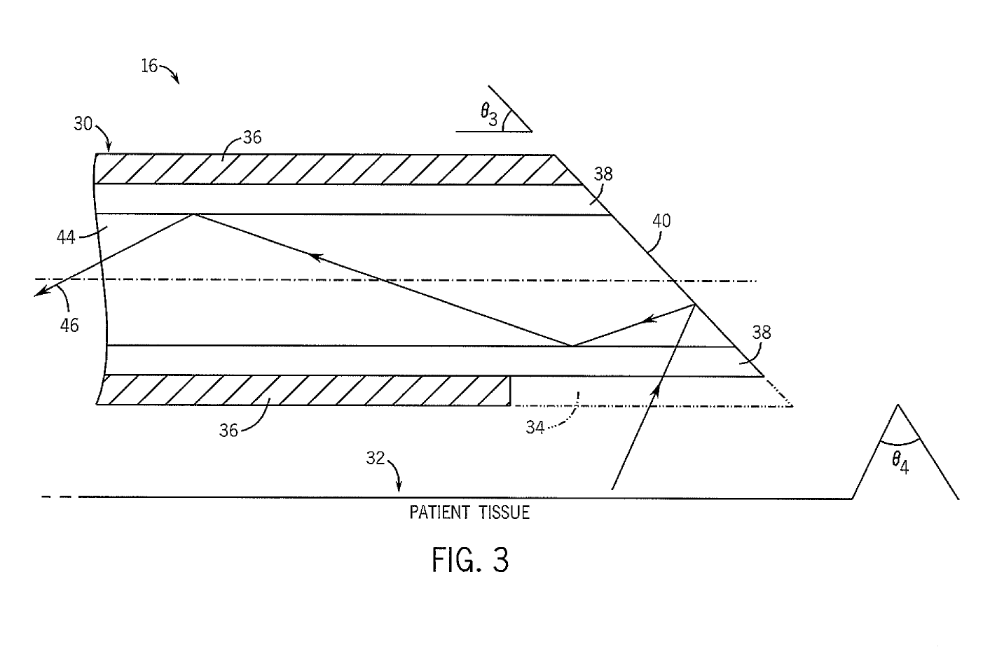

Turning to FIG. 3, the figure illustrates an embodiment of a terminal portion 16 of a fiber optic element 30 as may be used to collect or acquire light in one embodiment. In such an embodiment, the fiber optic element 30 may include a multi-mode plastic optical fiber having an angled terminal portion 16. In other embodiments, the sensor 12 may include a multi-mode optical fiber bundle, each fiber in the bundle having an angled terminal portion 16. In further embodiments, any class of optical fiber that is capable of transmitting suitable light wavelengths may be used, for example, single-mode fiber. The angled terminal portion 16 may be used to reflect light collected from the patient tissue 32 back into the fiber, such as toward a monitor 18 (FIG. 1), It is to be understood that the fibers used for light emission and the used for light collection may be of the same type, for example, multi-mode optical fiber, but they need not be. That is, the fibers may be matched for particular uses, for example, a single-mode light emitting fiber may be used in conjunction with a multi-mode light collecting fiber bundle, or vice versa. Further, in one embodiment, both light emission and light collection may be performed on a common fiber optic element having an angled terminal portion. However, in other embodiments, the light emission and light collection may be performed on separate fiber optic elements having respective angled terminal portions.

In one embodiment, the terminal portion 16 of the fiber optic element 30 may have an angle θ₃ of approximately 45°. However, in other embodiments, the angle θ₃ may be between 30° and 60°. The terminal portion 16 may be formed using one or more of the cutting techniques noted above, e.g., by use of ceramic scissors, an angled cutting jig, a laser cutter, a robotic fiber cutter, or other suitable fiber cutting techniques. In addition, as previously noted, the terminal portion 16 of the cut fiber may be polished or otherwise finished (such as by coating or capping the terminal portion 16 with a reflective layer) to enhance reflectivity of the boundary surface 40. By enhancing the reflectivity of the boundary surface 40, more light may be collected and reflect through the fiber optic element 30.

Further, as discussed above, a tissue facing portion of the fiber optic element 30 has a section 34 where the fiber's jacket 36 has been removed to expose the cladding 38. In one embodiment, the exposed cladding 38 may be placed up against the patient tissue 32 at section 34, physically contacting the patient tissue 32. In another embodiment, the exposed cladding 38 does not physically contact the patient tissue 32 and there is an air gap between the exposed cladding 38 and the patient tissue 32 at section 34. In one such embodiment, the removal of the section 34 of the fiber jacket 36 allows light to be collected from the patient tissue 32. The collected light may then pass through the cladding 38 at section 34 and into the fiber optic element 30.

FIG. 3 also illustrates an example path that a collected ray of light 46 may follow while traveling from the patient tissue 32 through the fiber's core 44 to a monitor 18 (shown in FIG. 1). The ray of light 46 may be collected from patient tissue 32 and may pass through the section 34 of the fiber that has the jacket 36 removed. The ray of light 46 may then refract slightly through the exposed cladding 38 as it enters into the core 44 and proceeds towards the boundary surface 40. The boundary surface 40 is angled at an angle of θ₃ such that the ray of light 46 is reflected through the core 44. The ray of light 46 may then travel through the core 44, reflecting multiple times off the cladding 38 as it proceeds to the monitor 18 (shown in FIG. 1).

Collected rays of light may enter the fiber optic element 30 from the patient tissue 32 an angle θ₄. The angle θ₄ may vary in embodiments having the boundary surface 40 coated or capped with a reflective layer and in embodiments having the boundary surface 40 left uncoated and uncapped, as discussed above. That is, in certain embodiments, angle θ₄ may be derived based on Snell's law of refraction, taking into account the refractive index of the core 44, the refractive index of the cladding 38, the refractive index of the environment (e.g., air) immediately adjacent to the terminal portion 16, and the refractive index of the patient tissue 32 (in embodiments where the exposed cladding 38 is physically contacting the patient). The angle θ₃ and/or the coating applied to the terminal portion 16 of fiber optic element 30 may be selected taking into account the law of reflection and/or Snell's law to provide a desired angle θ₄ by which light may be collected from the patient tissue 32.

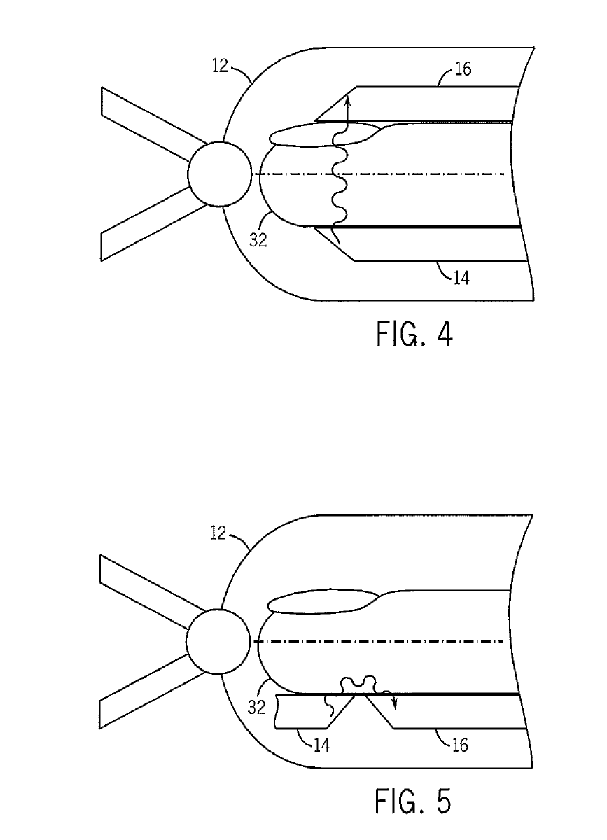

With the foregoing implementations of terminal portions of a fiber optic element in mind, FIGS. 4 and 5 illustrate transmissive and reflective modalities of a spectrophotometric sensor 12 (shown in FIG. 1). More specifically, FIG. 4 illustrates an implementation where a sensor is configured to transmit light emitted by a terminal portion 14 of a first fiber optic element toward a terminal portion 16 of a second fiber optic element positioned opposite the terminal portion 14 with respect to the tissue 32. In the depicted example, light emitting terminal portion 14 is placed on the tissue 32 opposite to light collecting terminal portion 16 so that light emitted by terminal portion 14 may be collected by terminal portion 16 after the light has passed through the blood perfused tissue 32. Each one of terminal portions 14, 16 may be placed so that the terminal portion physically contacts the patient tissue 32 or placed so that there is an air gap between the terminal portion and the patient tissue 32. In an alternative embodiment, the terminal portions 14, 16 may be secured to the patient tissue 32 by a bandage with stiffening elements to prevent the terminal portions from dislodging or otherwise moving out of position. However, in one embodiment, such as the depicted embodiment, the terminal portions 14, 16 may be secured to the patient tissue 32 by a clip-style holder or other rigid sensor body. Any structure that allows for a relatively secure attachment to the patient tissue 32 may be used to affix the terminal portions 14, 16.

FIG. 5 illustrates a reflectance implementation in which light emitted by a terminal portion 14 of a first fiber optic element is collected by a terminal portion 16 of a second fiber optic element positioned on the same side of the patient tissue 32. In the depicted embodiment, the light emitting terminal portion 14 is placed on the same surface of the tissue 32 as the light collecting terminal portion 16 so that the light emitted by terminal portion 14 may be collected by terminal portion 16 after the light has reflected and/or scattered through the blood perfused tissue 32. Each one of terminal portions 14 and 16 may be placed so that the terminal portion physically contacts the patient tissue 32 or may be placed so that there is a slight air gap between the terminal portion and the patient tissue 32. It is also to be understood that while the depicted embodiment shows the terminal portions 14 and 16 facing each other, other embodiments may include the two terminal portions 14 and 16 oriented parallel with each other and having the angled cut facing in the same direction.

In one embodiment, the terminal portions 14, 16 may be secured to the patient tissue 32 by a bandage with stiffening elements to prevent the terminal portions from dislodging or otherwise moving out of position. In other embodiments, such as the depicted embodiment, the terminal portions 14, 16 may be secured to the patient tissue 32 by a clip-style holder or other rigid sensor body. Any structure that allows for a relatively secure attachment to the patient tissue 32 may be used to affix the terminal portions 14, 16.

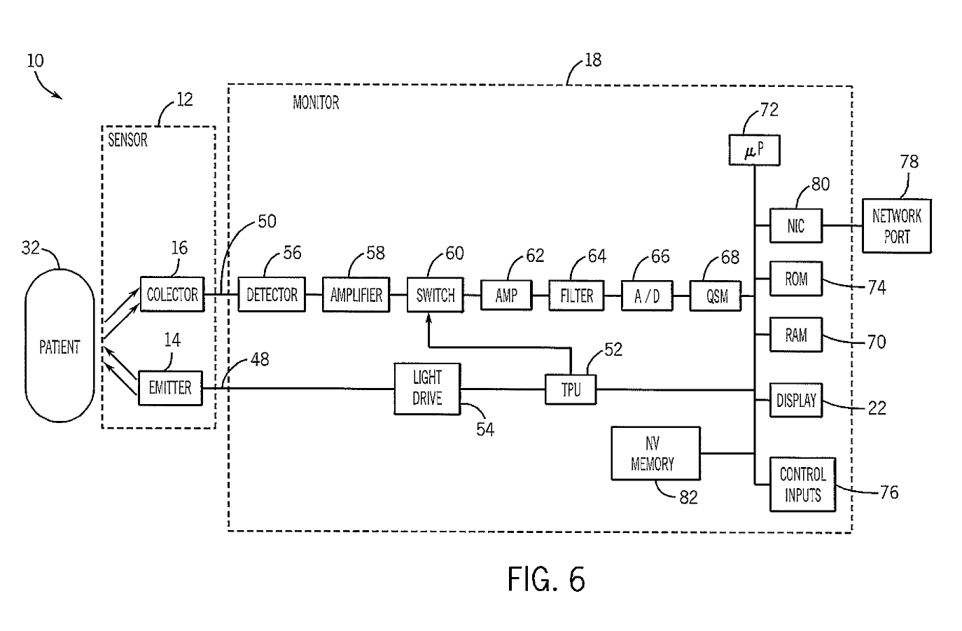

Turning to FIG. 6, the figure depicts a block diagram of an embodiment of the spectrophotometric system 10 that may be configured to implement the techniques described herein. By way of example, embodiments of the system 10 may be implemented with any suitable sensor and patient monitor, such as those available from Nellcor Puritan Bennett LLC. The system 10 may include the patient monitor 18 and the sensor 12 (as discussed with respect to FIG. 1), which may be configured to obtain, for example, a plethysmographic signal from patient tissue at certain predetermined wavelengths. Sensor 12 may include one or more optical fibers or fiber bundles, as discussed herein, in optical communication with the patient monitor 18. Embodiments of the sensor 12 may be free of electronics, metals, and/or other materials that may influence or be influenced by a magnetic field. Certain components of the sensor 12, namely a light emitting terminal portion 14 (i.e., an emitter or emitter region) and a light collecting terminal portion 16 (i.e., a detector or detector region) are illustrated in FIGS. 2, 3 above. Other components of the system 10 include a fiber optic element 48 through which light is transmitted from the monitor 18 to the light emitting terminal portion 14 and a fiber optic element 50 through which light is transmitted from the light collecting terminal portion 16 to the monitor 18 are also depicted. When the system 10 is in operation, light traveling through the fiber optic element 48 may be emitted by the light emitting terminal portion 14, may pass into the patient tissue 32, may be absorbed and/or scattered by the tissue, and may be collected by the light collecting terminal portion 16 to pass back to the monitor 18 along the fiber optic element 50.

In one embodiment, a time processing unit (TPU) 52 may provide timing control signals to light drive circuitry 54 within the monitor 18. Light drive circuity 54 may contain a set of emitters (e.g., LEDs) which may be capable of emitting light through fiber optic element 48. Light drive circuity 54 may control which wavelength of light is emitted by turning on a suitable LED configured to emit light near a certain wavelength. Light drive circuitry 54 may also control when light is emitted, and if multiple light sources are used, the multiplexed timing for the different light sources. Light emitted from the light drive circuitry 54 may be transmitted through fiber element or fiber bundle 48 to the sensor 12 and may be reflected out of the light emitting terminal portion 14 into the patient tissue 32 as described with respect to FIG. 2. The light may be absorbed and/or scattered by the tissue 32, and may be collected by the light collecting terminal portion 16. The light collecting terminal portion 16 may reflect the collected light through the fiber optic element or fiber optic bundles 50 to the detector 56 within the monitor 18, as described with respect to FIG. 3.

The TPU 52 may also control the gating-in of signals from the detector 56 through an amplifier 58 and a switching circuit 60. These signals may be sampled at the proper time, depending upon which of multiple light sources is illuminated, if multiple light sources are used. The received signals from the detector 56 may be passed through an amplifier 62, a low pass filter 64, and an analog-to-digital converter 66 for amplifying, filtering, and digitizing the received signals. The digital data may then be stored in a queued serial module (QSM) 68, for later downloading to the RAM 70 as the QSM 68 fills up. In an embodiment, there may be multiple parallel paths for separate amplifiers, filters, and A/D converters for multiple light wavelengths or spectra received. This raw digital diagnostic data may be further sampled by the circuitry of the monitor 18 into specific diagnostic data of interest, such as pulse rate, blood oxygen saturation, and so forth.

In various embodiments, based at least in part upon the value of the received signals corresponding to the light detected by detector 56, a microprocessor 72 may calculate a physiological parameter of interest using various algorithms. These algorithms may utilize coefficients, which may be empirically determined, corresponding to, for example, the wavelengths of light used. In one embodiment, these algorithms may be stored in the ROM 74.

The monitor 18 may include control inputs 76 such as a switch, dial, buttons, a keyboard, a mouse, a trackball, or a network port 78 providing instructions from a remote host computer communicating through a network interface card (NIC) 80. A display 22 may be used to show the physiological measurements, alarm limits, and other information of interest to a caregiver. Nonvolatile memory 82 may store caregiver preferences, patient information, or various parameters, discussed above, which may be used in the operation of the monitor 18. Software for performing the configuration of the monitor 18 and for carrying out the techniques described herein may also be stored on the nonvolatile memory 82, or may be stored on the ROM 74. The nonvolatile memory 82 and/or RAM 70 may also store historical values of various discrete medical diagnostic data points. By way of example, the nonvolatile memory 82 and/or RAM 70 may store values of corresponding to the pulse rate, blood oxygen saturation, and total hemoglobin, and others.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Indeed, the disclosed embodiments may not only be applied to measurements of blood oxygen saturation, but these techniques may also be utilized for the measurement and/or analysis of other blood constituents or other constituents suitable for spectrophotometric analysis. For example, using the same, different, or additional wavelengths, the present techniques may be utilized for the measurement and/or analysis of carboxyhemoglobin, met-hemoglobin, total hemoglobin, fractional hemoglobin, intravascular dyes, and/or water content. Rather, the various embodiments may cover all modifications, equivalents, and alternatives falling within the spirit and scope of the disclosure as defined by the following appended claims.

What is claimed is:

1. A spectrophotometric sensor, comprising:
a sensor body; and
at least one optically transmissive fiber disposed within the sensor body, wherein the optically transmissive fiber comprises an angled cut capable of reflecting light transmitted through the optically transmissive fiber outward from a patient facing surface or of reflecting light collected from the patient facing surface through the optically transmissive fiber.

2. The spectrophotometric sensor of claim 1, wherein at least one of the optically transmissive fibers comprises multi-mode fiber.

3. The spectrophotometric sensor of claim 1, wherein at least one of the optically transmissive fibers comprises single-mode fiber.

4. The spectrophotometric sensor of claim 1 wherein the angled cut is between about 30° and about 60°.

5. The spectrophotometric sensor of claim 1, comprising first and second optically transmissive fibers comprising the angled cut, wherein the first and the second optically transmissive fibers are positioned to be on opposite sides of a patient tissue when in use.

6. The spectrophotometric sensor of claim 1, comprising first and second optically transmissive fibers comprising the angled cut, wherein the first and the second optically transmissive fibers are positioned to be on the same side of a patient tissue when in use.

7. The spectrophotometric sensor of claim 1, wherein the sensor body comprises a clip-style sensor body.

8. The spectrophotometric sensor of claim 1, wherein the sensor body comprises a bandage-style sensor body comprising one or more stiffening elements.

9. The spectrophotometric sensor of claim 1, comprising a gel configured to be disposed at approximately the angled cut as a light coupling medium.

10. A monitoring system, comprising:
a monitor, comprising:
one or more light sources; and
one or more light detectors; and
a sensor, comprising:
a sensor body;
at least one light transmission fiber optically connected to the one or more light sources, the at least one light transmission fiber comprising a first angled terminal portion capable of reflecting light transmitted through the light transmission fiber toward a patient facing surface of the sensor body; and
at least one light reception fiber optically connected to the one or more light detectors, the at least one light reception fiber comprising a second angled terminal portion capable of reflecting light collected from the same or a different patient facing surface of the sensor body through the light reception fiber.

11. The system of claim 10, wherein the one or more light sources comprise one or more light emitting diodes.

12. The system of claim 10, wherein the monitor comprises a pulse oximeter.

13. The system of claim 10, wherein one or both of the first angled terminal portion or the second angled terminal portion comprise an angle of between about 30° and about 60°.

14. A method of manufacturing a sensor, comprising the acts of:
cutting an optical fiber at an angle such that the optical fiber comprises an angled terminus;
removing a portion of a jacket surrounding the optical fiber proximate to the angled terminus; and
positioning the optical fiber within a sensor body such that the angled terminus will reflect light toward a patient facing surface of the sensor body or will collect light emanating from the patient facing surface.

15. The method of claim 14, wherein the angled terminus is cut at an angle of between about 30° and about 60°.

16. The method of claim 14, wherein the angled terminus is cut at an angle of about 45°.

17. The method of claim 14, comprising coating the angled terminus with a reflective finish.

18. The method of claim 14, comprising capping the angled terminus with a mirror.

19. The method of claim 14, comprising polishing the angled terminus.

20. The method of claim 14, comprising disposing a gel at approximately the angled terminus, wherein the gel is configured as a light coupling medium.
